(19) United States
(12) Patent Application Publication
(10) Pub. No.: US 2024/0424049 A1
(43) Pub. Date: Dec. 26, 2024

(54) COMPOSITION FOR ALLEVIATING HAIR LOSS AND PROMOTING HAIR GROWTH COMPRISING OAT EXTRACT

(71) Applicants: KONKUK UNIVERSITY INDUSTRIAL COOPERATION CORP, Seoul (KR); REPUBLIC OF KOREA(MANAGEMENT : RURAL DEVELOPMENT ADMINISTRATION), Jeonju-si, Jeollabuk-do (KR)

(72) Inventors: Sun Hee DO, Hanamsi, Gyeonggi-do (KR); Woo-Duck SEO, Wanju-gun, Jeollabuk-do (KR); Hyosung KIM, Seoul (KR); Mi Ja LEE, Wanju-gun, Jeollabuk-do (KR); Hyun Young KIM, Wanju-gun, Jeollabuk-do (KR); Ki-Do PARK, Miryang-si, Gyeongsangnam-do (KR)

(21) Appl. No.: 18/697,432

(22) PCT Filed: Oct. 4, 2022
(86) PCT No.: PCT/KR2022/014896

§ 371 Date: Mar. 29, 2024

(30) Foreign Application Priority Data
Oct. 1, 2021 (KR) 10-2021-0131114

(51) Int. Cl. A61K 36/899; A61K 8/60; A61K 8/9794; A61K 31/7048; A61K 31/728; A61K 38/39; A61P 17/14; A61Q 7/00

(52) U.S. Cl. CPC A61K 36/899; A61K 8/602; A61K 8/9794; A61K 31/7048; A61K 31/728; A61K 38/39; A61P 17/14; A61Q 7/00; A61K 2236/333

(57) ABSTRACT

The present invention relates to a composition, a food composition, a cosmetic composition, and a feed composition for hair loss improvement or hair growth promotion, which contain oat young leaf and/or stem extract, and a pharmaceutical composition and a medical device for prevention or treatment of hair loss disease, which contain oat young leaf and/or stem extract.

The oat young leaf and/or stem extract of the present invention has an excellent hair loss improving and hair growth promoting effect by promoting the proliferation of HDP cells and the expression of related genes, and can thus be utilized as medicines, medical devices, feed, foods, health functional foods, cosmetics, and the like that are effective in prevention and treatment of hair loss.

### CROSS-REFERENCE TO RELATED APPLICATIONS

The present application is the 35 USC § 371 national phase of international application no. PCT/KR2022/014896, filed on Oct. 4, 2022, which claims the benefit of and priority to Korean Patent Application No. 10-2021-0131114, filed on Oct. 1, 2021. The contents of each are hereby incorporated by reference in their entireties.

### TECHNICAL FIELD

The present invention relates to a composition, a food composition, a cosmetic composition, and a feed composition for the improvement of hair loss or promotion of hair growth, which contain oat extract or a compound derived therefrom as an active ingredient, and a pharmaceutical composition, a treatment method, and a medical device for the prevention or treatment of hair loss disease, which contain oat extract or a compound derived therefrom as an active ingredient.

### BACKGROUND ART

Hair loss refers to the absence of hair in areas where hair should normally exist, and means the loss of hair on the scalp. In a 2019 survey, 29% of adult men and 15% of adult women responded that they were ‘currently experiencing hair loss symptoms’, and 49% of men and 27% of women in their 60s or older responded that they were experiencing hair loss symptoms. In addition, 47% of those currently experiencing hair loss symptoms felt that hair loss was severe, indicating a great need for hair loss treatment (Gallup Korea, survey on hair loss—2013/2019 comparison). To date, minoxidil, Propecia (finasteride), and Dutasteride have been approved as therapeutic agents for hair loss at home and abroad and are being used to treat patients with hair loss. However, the commercially available drugs Propecia and Dutasteride have been reported to have side effects such as erectile dysfunction, decreased libido, ejaculation disorders, breast tenderness and gynecomastia including breast enlargement, and have limitations in that they cannot be used by pregnant women or women who may be pregnant since they can cause abnormalities in the external genitalia of the male fetus in the case of being taken by a pregnant woman. In addition, when minoxidil is administered orally, side effects such as fluid retention, tachycardia, nausea, dyspnea, and gynecomastia in men have been reported, and side effects such as scalp irritation such as scalp dryness, itching, and erythema and allergic contact dermatitis have been reported when minoxidil is applied to the affected area for a long period of time (Recent Patents on Inflammation & Allergy Drug Discovery 6(2), 2012, 130-136). Moreover, minoxidil slows down the progression of hair loss, but it is difficult to expect the effect of long, thick hair growth, and it is difficult to expect satisfactory results in people who have completely lost hair. Therefore, in order to develop therapeutic agents for hair loss that are similar or superior to the existing approved therapeutic agents for hair loss in the effect and do not have side effects such as scalp irritation, dermatitis, or sexual dysfunction, studies using natural substances with relatively proven safety are actively underway.

Meanwhile, pulse sprouts, which are the above-ground part of pulse crops including young leaves or stems after sowing, for example, oat young leaves or stems have long been known as a nutritionally excellent food source having high contents of vitamins and minerals, and contain functional secondary metabolites that exhibit antioxidant effects, and their industrial applicability as a health functional food and a pharmaceutical material is increasing.

### DISCLOSURE

### Technical Problem

Against this background, the present inventors have found out that oat young leaf extract or a compound derived therefrom has significant hair loss improvement and hair growth promotion efficacy and thus completed the present invention.

### Technical Solution

An object of the present invention is to provide a composition for hair loss improvement or hair growth promotion containing oat extract or a compound derived therefrom as an active ingredient.

Another object of the present invention is to provide a food composition for hair loss improvement or hair growth promotion containing oat extract or a compound derived therefrom as an active ingredient.

Still another object of the present invention is to provide a cosmetic composition for hair loss improvement or hair growth promotion containing oat extract or a compound derived therefrom as an active ingredient.

Still another object of the present invention is to provide a feed composition for hair loss improvement or hair growth promotion containing oat extract or a compound derived therefrom as an active ingredient.

Still another object of the present invention is to provide a pharmaceutical composition for prevention or treatment of hair loss disease containing oat extract or a compound derived therefrom as an active ingredient.

Still another object of the present invention is to provide a medical device for prevention or treatment of hair loss disease containing oat extract or a compound derived therefrom as an active ingredient.

Still another object of the present invention is to provide a use for hair loss improvement or hair growth promotion of a composition containing oat extract or a compound derived therefrom as an active ingredient.

Still another object of the present invention is to provide a use for prevention or treatment of hair loss disease of a composition containing oat extract or a compound derived therefrom as an active ingredient.

Still another object of the present invention is to provide a method for preventing or treating hair loss disease, the method including administering a composition containing oat extract or a compound derived therefrom as an active ingredient to a subject with hair loss disease.

### Advantageous Effects

The oat extract of the present invention has an excellent hair loss improving and hair growth promoting effects by increasing the growth rate of human-derived dermal papilla cells, and can be thus utilized as medicines, foods, health functional foods, cosmetics, medical devices and the like that are effective in prevention and treatment of hair loss.

### BRIEF DESCRIPTION OF DRAWINGS

FIG. 1 illustrates the results of a cytotoxicity test of oat young leaf extract;

FIGS. 2A and 2B are diagrams illustrating the formation of HDP spheroids to examine the effect of oat young leaf extract or compounds derived therefrom;

FIGS. 3A and 3B are diagrams illustrating the hair follicle cell proliferation activity in terms of diameter and genomic DNA concentration to examine the effect of oat young leaf extract or compounds derived therefrom;

FIG. 4 is a diagram illustrating the analysis results of expression of genes related to HDP cell proliferation to examine the effect of oat young leaf extract;

FIGS. 5A to 5C are diagrams illustrating the analysis results of expression levels of genes related to hair follicle cell proliferation to examine the effect of oat young leaf extract;

FIG. 6 is a diagram illustrating the analysis results of expression levels of genes related to hair cycling to examine the effect of oat young leaf extract; and

FIG. 7 is a diagram illustrating the analysis results of expression of growth factors related to hair follicle formation and regeneration to examine the effect of oat young leaf extract.

### DETAILED DESCRIPTION OF PREFERRED EMBODIMENTS

The present invention is explained in detail as follows. Meanwhile, each description and each embodiment disclosed in the present invention may also be applied to other descriptions and other embodiments, respectively. In other words, all combinations of various elements disclosed in the present invention fall within the scope of the present invention. Additionally, the scope of the present invention is not limited to the specific description described below.

Additionally, those skilled in the art will recognize, or be able to ascertain using no more than routine experimentation, a number of equivalents to the specific aspect of the present invention described herein. In addition, such equivalents are intended to be included in the present invention.

An aspect of the present invention for achieving the objects provides a composition for hair loss improvement or hair growth promotion containing oat extract or a compound derived therefrom as an active ingredient.

Another aspect of the present invention provides a food composition for hair loss improvement or hair growth promotion containing oat extract or a compound derived therefrom as an active ingredient.

Still another aspect of the present invention provides a cosmetic composition for hair loss improvement or hair growth promotion containing oat extract or a compound derived therefrom as an active ingredient.

Still another aspect of the present invention provides a feed composition for hair loss improvement or hair growth promotion containing oat extract or a compound derived therefrom as an active ingredient.

In the present invention, the oat extract may not mean an extract of oat seeds, but may mean an extract of oat young leaves, oat stems, or a combination thereof, and as an example, the oat extract may be oat young leaf extract or may be oat young leaf and stem extract extracted from oat stems in addition to oat young leaves.

The term “oat young leaves” refers to young plants of oats, a type of pulse crop, that have sprouted and grown to approximately 10 to 40 cm. Typically, the oat young leaves can be obtained by immersing oats in water for about one day, removing moisture from the oats, sowing and germinating the oats under a condition to prevent moisture loss, then growing the sprouts for about 7 to 15 days when grown in facilities, or for about 25 to 35 days when grown in open fields after sowing.

In the present invention, as the oat young leaves, those commercially available may be purchased and used, or those collected from nature or cultivated may be used, but the oat young leaves are not limited thereto.

In the present invention, it has been found that when HDP (human dermal papilla) cells are treated with oat young leaf and/or stem extract or a compound derived therefrom, expression of genes related to HDP cell proliferation, hair follicle cell proliferation, or hair cycling is promoted, and in particular, the effect is similar or superior to that of minoxidil, which is commercially available as a therapeutic agent for hair loss, and it has been thus discovered for the first time that oat young leaf extract or a compound derived therefrom can be utilized to improve hair loss or promote hair growth.

The oat young leaf and/or stem extract of the present invention refers to a form of extract obtained through extraction using one or more solvents, and as the solvent used in the preparation of the extract, water, an alcohol having 1 (C₁) to 6 (C₆) carbon atoms, specifically, ethanol, methanol or butanol, an organic solvent, or any mixed solvent thereof may be used, but the solvent is not limited thereto. As the extraction method, solvent extraction, hot water extraction, cold extraction, reflux cooling extraction, ultrasonic extraction, or steam extraction may be used, but the extraction method is not limited thereto.

The extract may be prepared and used in a dry powder form after extraction, but is not limited thereto.

In a specific embodiment of the present invention, water and alcohol have been added to oat young leaves or stems crushed into powder to obtain oat young leaf or stem extract.

As used herein, the term “compound” refers to a compound isolated or purified from oat young leaf or stem extract. Specifically, the compound may be a compound represented by any one of the following Chemical Formulas: Chemical Formula 1 (avenacoside B), Chemical Formula 2 (26-desglucoavenacoside B), and Chemical Formula 3 (isovitexin 2″-O-arabinoside).

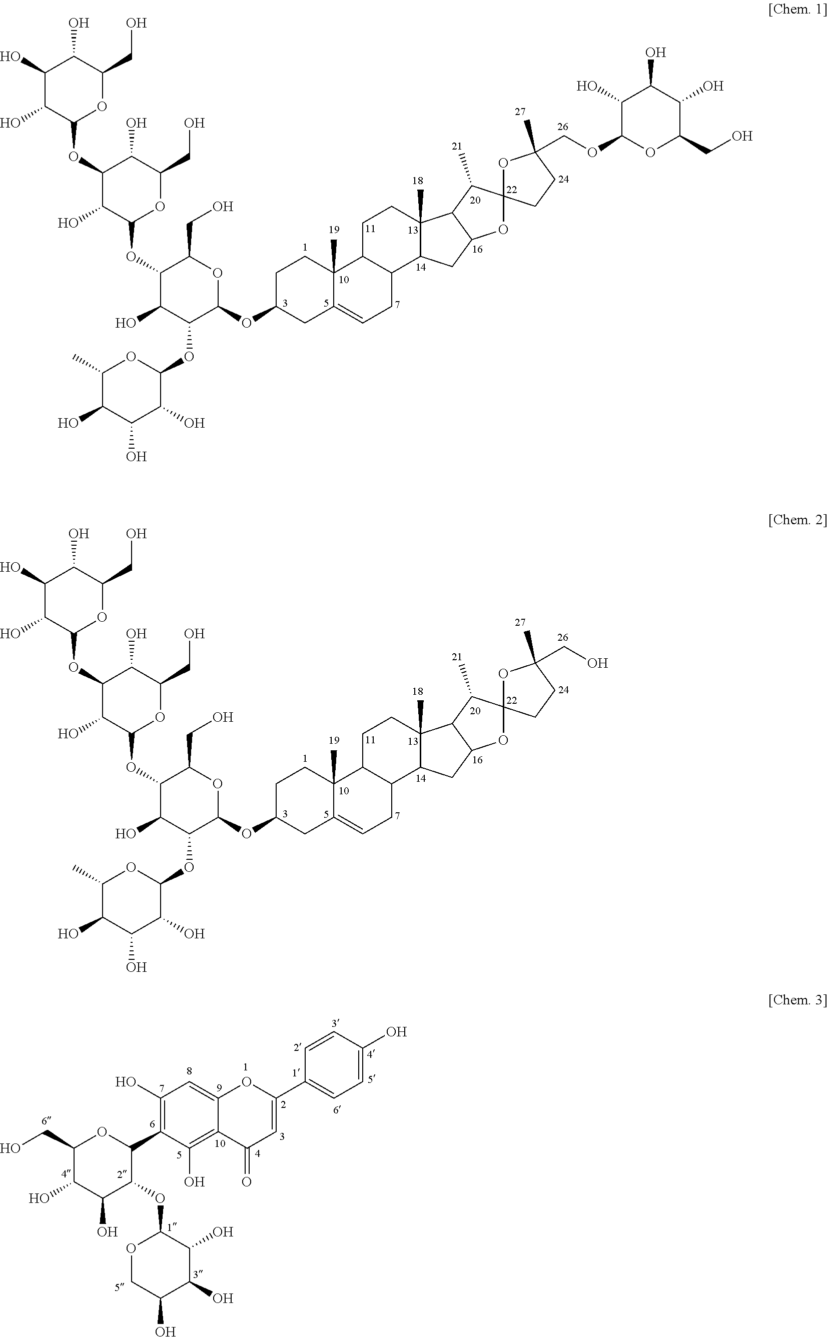

Herein “hair loss improvement” comprehensively refers to the process of treating, alleviating, or relieving the hair loss state or an effect thereof, and may refer to any action that inhibits the progression of hair loss. Herein “hair growth promotion” refers to any action that increases hair growth from hair follicles, and may refer to any action that increases the total amount of hair. The hair loss improvement or hair growth promotion may be achieved by promoting the expression of genes related to HDP (human dermal papilla) cell proliferation, hair follicle cell proliferation, or hair cycling.

When HDP (human dermal papilla) cells are treated with the oat young leaf and/or stem extract or a compound derived therefrom of the present invention, it has been found that the expression of genes related to HDP (human dermal papilla) cell proliferation, hair follicle cell proliferation, or hair cycling is promoted, and in particular, the effect is similar or superior to that of minoxidil, which is commercially available as a therapeutic agent for hair loss, and oat young leaf extract or a compound derived therefrom can be utilized to improve hair loss or promote hair growth.

HDP (human dermal papilla) cells are one of the cells around hair follicles/hair roots, and are one of the hair matrix cells that produce hair.

In a specific embodiment of the present invention, when HDP (human dermal papilla) cells are treated with the oat young leaf and/or stem extract or a compound derived therefrom, it has been found that the expression of genes related to HDP (human dermal papilla) cell proliferation, hair follicle cell proliferation, or hair cycling is promoted, and in particular, the effect is similar or superior to that of minoxidil, which is commercially available as a therapeutic agent for hair loss, and it has been verified that oat young leaf extract or a compound derived therefrom can be utilized to improve hair loss or promote hair growth.

The composition of the present invention may be a food composition, cosmetic composition, or feed composition. The composition may further contain a pharmaceutically acceptable carrier, and may be formulated with the carrier and provided as foods, cosmetics, and feed.

Herein “food” includes all foods in the conventional sense such as meat, sausages, bread, chocolate, candies, snacks, confectionery, pizza, ramen, other noodles, gum, dairy products including ice cream, various soups, beverages, tea, drinks, alcoholic beverages, vitamin complexes, and health functional foods. The food is not limited thereto, as long as it can contain the oat young leaf extract of the present invention. The food may be prepared by adding the oat young leaf extract of the present invention to juice, tea, jelly, juice, and the like, and the food composition of the present invention may be prepared in the form of a powder, granule, tablet, capsule, pill, or liquid, but is not limited thereto.

At the time of preparation, the food composition may be prepared by adding raw materials and ingredients commonly added in the art, and the types are not particularly limited. For example, like common foods, the food composition may contain additional ingredients such as various herbal extracts, sitologically acceptable food supplements, or natural carbohydrates, but is not limited thereto.

Specifically, additives that can be added may be hyaluronic acid, collagen, or a combination thereof, but are not limited thereto.

In a case where the food composition of the present invention is a health functional food composition, the “health functional food” refers to food prepared and processed using raw materials or ingredients, which have functional properties useful to the human body in accordance with Act No. 6727 on Health Functional Food, and ‘functionality’ refers to obtaining useful effects for health purposes such as nutrient control or physiological action on the structure and function of the human body. Meanwhile, health foods refer to foods that have a more active health maintaining or promoting effect than general foods, and health supplement foods refer to foods for health supplement purposes. In some cases, the terms health functional food, health food, and health supplement may be used interchangeably.

The food composition of the present invention may be added as it is or used together with other foods or food ingredients, and may be used appropriately according to conventional methods.

The health functional food composition of the present invention can be prepared by methods commonly used in the art, and may be prepared by adding raw materials and ingredients commonly added in the art. Specifically, the health functional food composition may further contain a physiologically acceptable carrier, the type of which is not particularly limited, and any carrier commonly used in the art may be used.

The health functional food composition may contain food additives such as preservatives, disinfectants, antioxidants, colorants, coloring agents, bleaching agents, seasonings, sweeteners, flavors, leavening agents, fortifying agents, emulsifiers, thickening agents, coating agents, gum base agents, defoaming agents, solvents, and improving agents. The additives may be selected depending on the type of food and used in an appropriate amount. The health functional food may be prepared in any formulation without limitation as long as it is a formulation recognized as a food.

As used herein, the term “cosmetic composition” may be prepared in a formulation selected from the group consisting of a solution, an external ointment, a cream, a foam, a nourishing lotion, a softening lotion, a pack, a softening water, a milky lotion, a makeup base, an essence, a liquid cleanser, a bath additive, a sunscreen cream, a sun oil, a suspension, an emulsion, a paste, a gel, a lotion, a powder, a soap, a surfactant-containing cleanser, an oil, a powder foundation, an emulsion foundation, a wax foundation, a patch, and a spray, and may be specifically prepared in the form of an external preparation for skin, but is not limited thereto.

In the present invention, the external preparation for skin may be one selected from the group consisting of a hair tonic, a hair cream, a hair lotion, a hair shampoo, a hair rinse, a hair conditioner, a hair spray, a hair aerosol, pomade, a powder, a gel, a hair pack, a hair treatment, an eyebrow hair growth agent, an eyelash growth agent, and an eyelash nutritional agent, but is not limited thereto.

The cosmetic composition of the present invention may further contain one or more cosmetically acceptable carriers that are blended with general skin cosmetics, and common ingredients, for example, oil, water, surfactants, moisturizers, lower alcohols, thickeners, chelating agents, colorants, preservatives, and flavors may be appropriately blended, but the cosmetically acceptable carriers are not limited thereto. The cosmetically acceptable carriers contained in the cosmetic composition of the present invention vary depending on the formulation.

The cosmetic composition of the present invention may contain commonly used auxiliary agents, for example, hydrophilic or lipophilic gelling agents, hydrophilic or lipophilic active agents, preservatives, antioxidants, solvents, fragrances, fillers, blocking agents, pigments, odorants and dyes in addition to the oat young leaf extract. The mixing amount of the active ingredient contained in the cosmetic composition of the present invention may be appropriately determined depending on the purpose of use.

As used herein, the term “feed” refers to any natural or artificial diet, meal, or the like or a component of the meal, for or proper for eating, ingestion, and digestion by animals, and the feed containing the oat young leaf extract of the present invention can be prepared in various forms of feed known in the art and may specifically include concentrated feed, roughage, or special feed. The subject of breeding may include any living organism that can consume the feed of the present invention without limitation.

For administration of the feed composition of the present invention, additionally one or more selected from organic acids such as citric acid, fumaric acid, adipic acid, and lactic acid: phosphates such as potassium phosphate, sodium phosphate, and polymerized phosphates; and natural antioxidants such as polyphenol, catechin, tocopherol, vitamin C, green tea extract, chitosan, and tannic acid may be mixed and used, and if necessary, other common additives such as anti-influenza agents, buffer solutions, and bacteriostatic agents may be added. The feed composition may be prepared into injectable formulations such as aqueous solutions, suspensions, and emulsions, capsules, granules or tablets by additionally adding diluents, dispersants, surfactants, binders or lubricants.

The feed composition of the present invention may be used together with nutritional supplements, growth promoters, digestion and absorption promoters, and disease preventive agents in addition to various adjuvants such as amino acids, mineral salts, vitamins, antioxidants, antifungal agents, and antibacterial agents as auxiliary ingredients and main ingredients such as vegetable protein feed such as pulverized or crushed wheat, barley, and com, animal protein feed such as blood meal, meat meal, and fish meal, animal fat, and vegetable fat.

The feed composition containing the oat young leaf and/or stem extract or a compound derived therefrom as an active ingredient of the present invention can be used to improve hair loss or promote hair growth in breeding subjects.

Another aspect of the present invention provides a pharmaceutical composition for prevention or treatment of hair loss disease containing oat extract or a compound derived therefrom as an active ingredient.

Still another aspect of the present invention provides a method for preventing or treating hair loss disease, the method including administering the pharmaceutical composition to a subject with hair loss disease.

Still another aspect of the present invention provides a medical device for prevention or treatment of hair loss disease containing oat extract or a compound derived therefrom as an active ingredient.

The “oat extract” and “compound derived therefrom” of the present invention are as described above.

Hair loss of the present invention refers to the absence of hair in areas where hair should normally exist, and may specifically mean the loss of hair (thick, black hair) on the scalp. The causes of hair loss are not limited, but include genetic causes, hormonal imbalance, mental stress, exposure to air pollution, various eating habits such as consumption of processed foods, and environmental influences. For example, the hair loss includes hereditary androgenetic alopecia (baldness), alopecia areata, tinea capitis due to fungal infection, telogen effluvium, hair-pulling disorder, hair production disorder, and the like, and cicatricial alopecia, in which scars are formed, may mean alopecia due to lupus, toxic folliculitis, lichen planus pilaris, burns, and trauma.

As used herein, the term “treatment” refers to clinical intervention to alter the natural processes of the subject or cell to be treated, and this may be done during the progression of a clinical pathological state or to prevent it. The intended therapeutic effect includes preventing the occurrence or recurrence of the disease, alleviating symptoms, reducing all direct or indirect pathological consequences of the disease, preventing metastasis, reducing the rate of disease progression, alleviating or temporarily relieving the disease state, remission, or improving prognosis. In the present invention, the intended therapeutic effect includes any action that improves the course of hair loss by administering the composition containing oat young leaf extract as an active ingredient. As used herein, the term “prevention” refers to any action that inhibits or delays the onset of the hair loss disease by administering the composition containing oat young leaf extract as an active ingredient according to the present invention.

The pharmaceutical composition of the present invention may exhibit a hair loss disease preventing or treating effect by promoting the expression of genes related to HDP cell proliferation, hair follicle cell proliferation, or hair cycling when HDP (human dermal papilla) cells are treated with the pharmaceutical composition.

As used herein, the term “pharmaceutical composition” may further contain appropriate carriers, excipients, or diluents commonly used in the preparation of pharmaceutical compositions. The composition containing a pharmaceutically acceptable carrier may be various oral or parenteral formulations. In the case of being formulated into preparations, the preparations may be prepared using commonly used diluents or excipients such as fillers, extenders, binders, wetting agents, disintegrants, and surfactants. Solid preparations for oral administration may include tablets, powders, granules, capsules, and the like, and these solid preparations may be prepared by mixing one or more compounds with at least one or more excipients, for example, starch, calcium carbonate, sucrose or lactose, and gelatin. In addition to simple excipients, lubricants such as magnesium stearate and talc may also be used. Liquid preparations for oral administration include suspensions, oral solutions, emulsions, and syrups, and may contain various excipients such as wetting agents, sweeteners, fragrances, and preservatives in addition to the commonly used simple diluents such as water and liquid paraffin. Preparations for parenteral administration may include sterilized aqueous solutions, non-aqueous solvents, suspensions, emulsions, freeze-dried preparations, and suppositories. As non-aqueous solvents and suspending agents, propylene glycol, polyethylene glycol, vegetable oil such as olive oil, injectable esters such as ethyl oleate, and the like may be used. As a base for suppositories, witepsol, macrogol, tween 61, cacao, laurinum, glycerogelatin, and the like may be used.

The pharmaceutical composition of the present invention is not limited thereto, but may have any one formulation selected from the group consisting of tablets, pills, powders, granules, capsules, suspensions, oral solutions, emulsions, syrups, sterilized aqueous solutions, non-aqueous solvents, suspensions, emulsions, freeze-dried preparations, and suppositories.

As used herein, the term “subject” refers to all animals such as monkeys, cows, horses, sheep, pigs, chickens, turkeys, quail, cats, dogs, mice, rats, rabbits or guinea pigs, including humans, that have developed or may develop the hair loss disease, and the diseases may be effectively prevented or treated by administering the pharmaceutical composition of the present invention to a subject. The pharmaceutical composition of the present invention may be administered in combination with existing therapeutic agents.

As used herein, the term “administration” means supplying a predetermined substance to a patient by any suitable method, and the composition of the present invention may be administered through any general route as long as it can reach the target tissue. The administration may be intraperitoneal administration, intravenous administration, intramuscular administration, subcutaneous administration, intradermal administration, oral administration, topical administration, intranasal administration, intrapulmonary administration, or rectal administration, but is not limited thereto. The pharmaceutical composition of the present invention may be administered by an arbitrary device capable of transporting the active substance to target cells. Preferred administration methods and preparations include intravenous injection, subcutaneous injection, intradermal injection, intramuscular injection, drip injection and the like. Injections may be prepared using aqueous solvents such as physiological saline solution and Ringer's solution and non-aqueous solvents such as vegetable oil, higher fatty acid esters (for example, ethyl oleic acid), and alcohols (for example, ethanol, benzyl alcohol, propylene glycol, and glycerin), and can contain pharmaceutical carriers such as stabilizers (for example, ascorbic acid, sodium bisulfite, sodium pyrosulfite, BHA, tocopherol, and EDTA) to prevent degeneration, emulsifiers, buffering agents for pH adjustment, and preservatives (for example, phenylmercuric nitrate, thimerosal, benzalkonium chloride, phenol, cresol, and benzyl alcohol) to inhibit microbial growth.

As used herein, the term “therapeutically effective amount” used in association with an active ingredient means the amount of oat young leaf and/or stem extract, a compound derived therefrom, or a pharmaceutically acceptable salt of these compounds that is effective in prevention or treatment of the target disease.

As used herein, the term “medical device” refers to medical equipment or a medical instrument; may mean a cream, lotion, or microneedle patch related to medical care in the present invention; and may include without limitation any medical device that those skilled in the art would readily consider.

### MODE FOR CARRYING OUT THE INVENTION

Hereinafter, the present invention will be described in more detail with reference to Examples. Examples are for illustrating the present invention more specifically, and the scope of the present invention is not limited to Examples.

### Example 1: Preparation of Oat Young Leaf Extract

Oats were sown and grown in a greenhouse at a temperature of 18° C. to 20° C. for about 10 to 15 days, and oat young leaves were harvested. The harvested oat young leaves were dried in the shade, cut into pieces, then immersed in 20% to 80% fermented alcohol for extraction, and the liquid component was obtained through filtration and freeze-dried, thereby preparing oat young leaf extract.

### Example 2: Identification of Compounds Derived From Oat Young Leaf Extract

The dried powder of the oat young leaf extract prepared in Example 1 was subjected to extraction using n-hexane (2×15, 24 hours each) at room temperature to remove lipids. After the remaining n-hexane was removed, extraction was performed using methanol at room temperature, and the liquid component was obtained through filtration and then concentrated under reduced pressure, thereby obtaining a crude extract of oat young leaves.

The crude extract (60.2 g) was dissolved in distilled water and fractionated using diethyl ether and n-butanol (3×2 L). The obtained water fraction (32.4 g) was fractionated using MeOH/H₂O as a developing solvent at a ratio of 1:9, 2:8, 3:7, 4:6, 5:5, 7:3, or 10:0 (v/v) by reversed-phase open column chromatography (Cosmosil 75C18-OPN) to obtain 11 fractions.

Fraction 2 (12.0 g) was further fractionated using MeOH/H₂O at a ratio of 5:1 to 1:3 (v/v) to obtain fractions 2-1 to 2-5. The subfraction 2-3 was fractionated using MeOH/H₂O at a ratio of 5:1 to 1:3 (v/v) by Cosmosil 75C18-OPN to obtain eight fractions, fractions 2-3-1 to 2-3-8. The subfractions 2-3-4 and 2-3-5 were further fractionated using MeOH/H₂O at a ratio of 3:1 to 1:1 (v/v) by Cosmosil 75C18-OPN to obtain compound 5 (35.0 mg) (Chemical Formula 2). The subfraction 2-3-8 was fractionated using MeOH/H₂O at a ratio of 5:1 to 1:3 (v/v) by reverse-phase column chromatography to obtain compound 4 (44.9 mg) (Chemical Formula 1). After subfractions 9-1 to 9-6 were obtained from the fraction 9 (8.2 g), compound 9 (365.6 mg) (Chemical Formula 3) was obtained from the subfraction 9-3 (2.4 g).

### Example 3: Cytotoxicity Assay

As HDP (human dermal papilla) cells, primary cells are commonly used, and recently, immortalized cell lines transfected with human telomerase transcriptase (hTERT) have become available, and the experiment was conducted using hTERT-HDP cells in the present experiment.

Specifically, the proliferation rate of HDP cells and the cytotoxicity of oat young leaf extract (OSE) to HDP cells were evaluated through CCK-8 assay. Specifically, the Cell count kit-8 (CCK-8) assay is a method in which formazan is formed through reduction of tetrazolium salt by intracellular dehydrogenase and cell viability is examined through absorbance analysis.

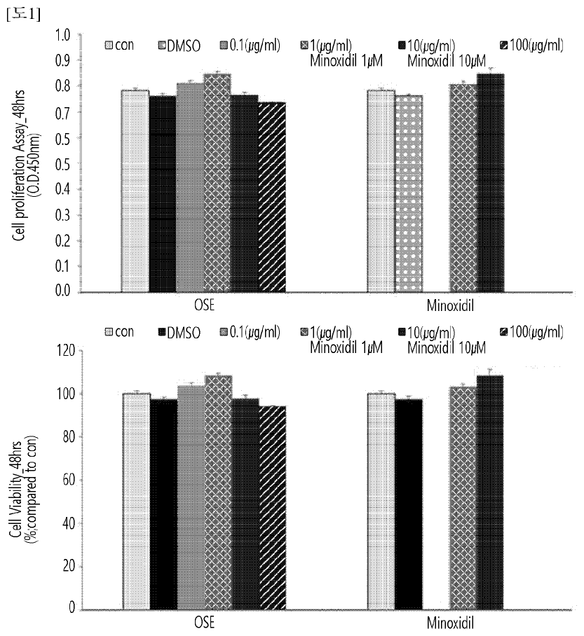

As a result, as can be seen from FIG. 1, it has been found that the oat young leaf extract does not exhibit cytotoxicity to HDP cells.

### Example 4: HDP Cell Proliferation Activity Analysis

HDP cells exhibit characteristics similar to those in vivo in 3D (spheroid) culture. Therefore, taking this into consideration, the cells were cultured in aggrewell800_24 well plate (300 microwells per well, microwell-800 um in size), and the effect of oat young leaf extract and compounds derived therefrom on spheroid formation was evaluated.

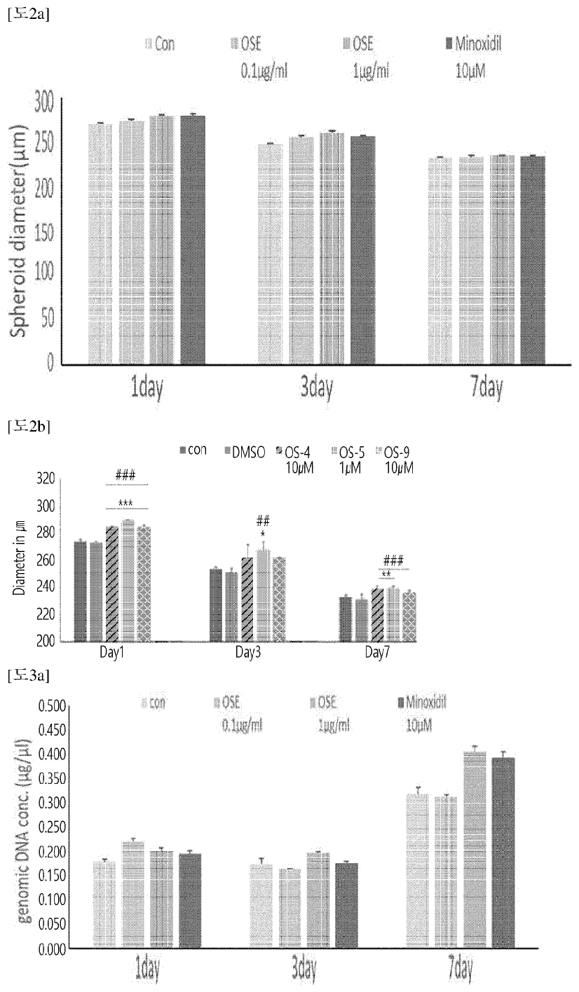

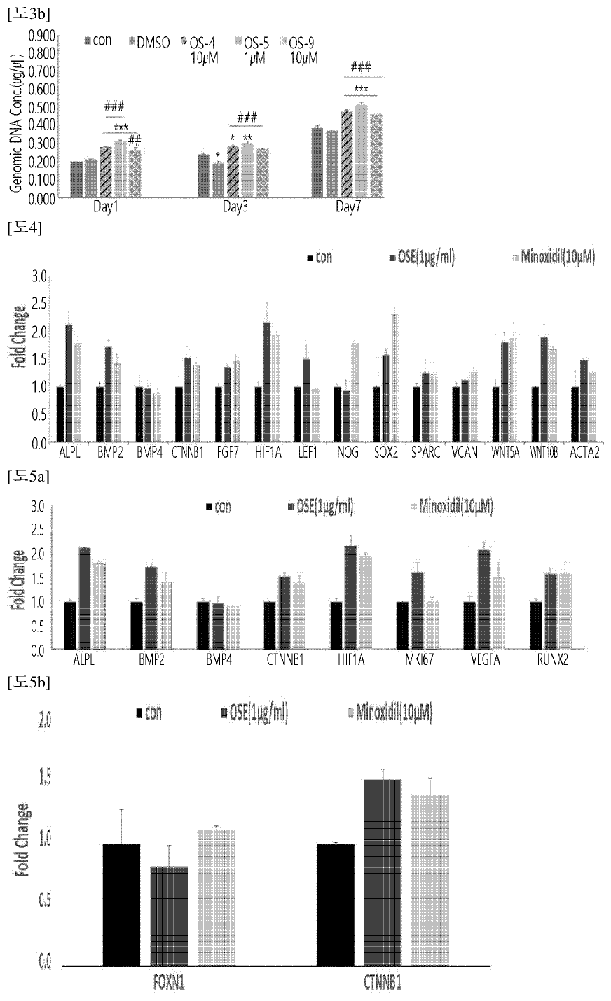

As a result, as can be seen from FIGS. 2 and 3, it has been found that the spheroid diameter (FIGS. 2A and 2B) gradually decreases in all groups over time from Day 1 to Day 7 but the genomic DNA concentration (FIGS. 3A and 3B) extracted from each spheroid significantly increases over time from Day 1 to Day 7 by the analysis.

This indicates that apoptosis and cell proliferation are occurring together, and as shown in the genomic DNA concentration results on Day 7, it has been found that HDP cell proliferation increases in the group treated with the oat young leaf extract or a compound derived therefrom of the present invention compared to that in the group treated with minoxidil, a positive control group.

It can be seen that particularly compound 4, avenacoside B (Chemical Formula 1), compound 5, 26-desglucoavenacoside B (Chemical Formula 2), and compound 9, isovitexin 2″-O-arabinoside (Chemical Formula 3) among the compounds have effects similar or superior to that of minoxidil.

### Example 5: Analysis of Expression of Genes Related to HDP Cell Proliferation

In order to evaluate the effect of the oat young leaf extract and compounds derived therefrom of the present invention on the expression of genes of HDP cells related to hair growth promotion, the expression level of genes related to hair growth promotion was analyzed by multiplex RT-PCR using the Day 7 cell samples recovered in the spheroid formation experiment in Example 4.

As a result, as can be seen from FIG. 4, when most genes known as HDP signature genes were treated with the oat young leaf extract (OSE) or a compound derived therefrom of the present invention, an increase in expression similar to that in the case of applying minoxidil as a positive control group was found. In particular, it has been found that the expression of CTNB1 (beta-catenin) and WNT5A as well as BMP2, FGF7, NOG, and VCAN (versican) among these was increased more than that in the case of applying minoxidil.

### Example 6: Analysis of Expression of Genes Related to Hair Follicle Cell Proliferation

In order to examine the effect of the oat young leaf extract and compounds derived therefrom of the present invention on the expression levels of genes related to the proliferation and differentiation of hair follicle cells, the expression levels of genes related to hair follicle cell proliferation were analyzed by multiplex RT-PCR using the Day 7 cell samples in a similar manner to that in Example 5.

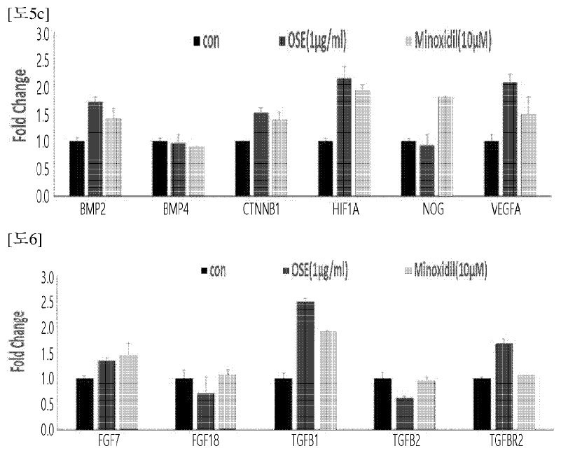

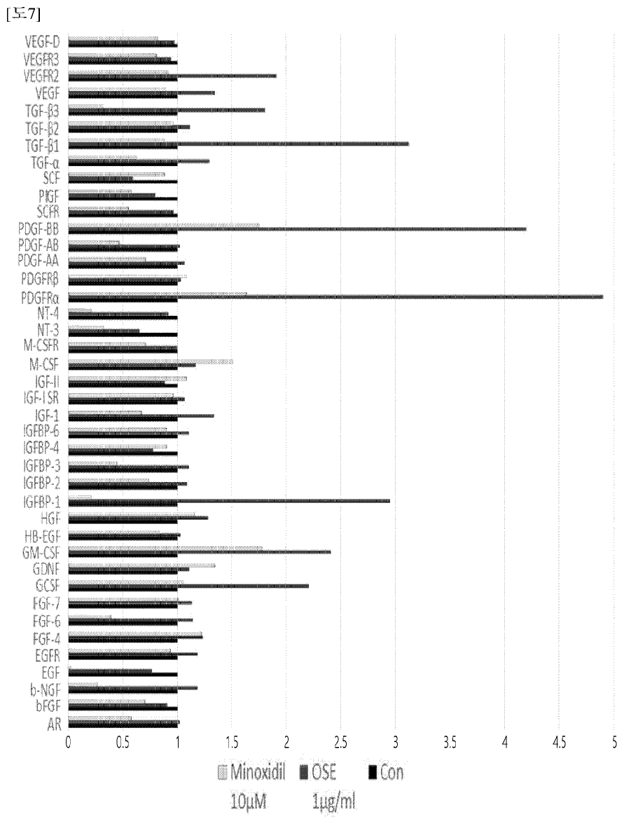

As a result, as can be seen from FIGS. 5A to 5C, it has been found that the expression levels of all genes are similar to those in the case of applying minoxidil, a positive control group, and among these, the expression of ALP, BMP2, beta-catenin (CTNB1), HIF1-alpha, Ki67 (MKI67), and VEGF was increased compared to that in the case of applying minoxidil.

### Example 7: Analysis of Expression Levels of Genes Related to Hair Cycling

In order to examine the effect of the oat young leaf extract and compounds derived therefrom of the present invention on the expression levels of genes related to hair cycling, the expression levels of genes related to hair cycling were analyzed by multiplex RT-PCR using the Day 7 cell samples in a similar manner to that in Example 5.

As a result, as can be seen from FIG. 6, it has been found that the expression of most genes was increased compared to that in the case of applying minoxidil, a positive control group, and particularly the increase in expression of TGF-beta1 was noticeable among these.

### Example 8: Analysis of Change in Expression of Growth Factor Related to Hair Follicle Formation and Regeneration

In order to examine the effect of the oat young leaf extract and compounds derived therefrom of the present invention on the degree of change in the expression of growth factors related to the hair follicle formation and regeneration, the degree of change in the expression of growth factors related to the hair follicle formation and regeneration was analyzed using the Day 7 cell samples in a similar manner to that in Example 5.

As a result, as can be seen from FIG. 7, it has been found that the expression of most genes was increased compared to that in the case of applying minoxidil, a positive control group, or most genes were expressed at a similar level to that in the case of applying minoxidil.

In summary, it can be seen that oat extract obtained from oat young leaves and/or stems has excellent effects in improving hair loss and promoting hair growth.

Based on the above description, those skilled in the art to which the present invention pertains will understand that the present invention may be implemented in a different specific form without changing the technical spirit or essential characteristics thereof. Therefore, it should be understood that the embodiments described above are not limitative, but illustrative in all respects. The scope of the present invention is defined by the appended claims rather than by the description preceding them, and therefore all changes or modified forms derived from the meaning and scope of the claims and their equivalent concepts should be construed as being included in the scope of the present invention.

1. A method for hair loss improvement or hair growth promotion, the method comprising the step of administering a composition comprising oat extract or a compound derived therefrom as an active ingredient to a subject.

2. The method according to claim 1, wherein the oat extract is extracted from oat young leaves, oat stems, or a combination thereof.

3. The method according to claim 1, wherein the extract is prepared through extraction using water, an alcohol having 1 to 6 carbon atoms, an organic solvent, or a mixed solvent thereof.

4. The method according to claim 1, wherein the compound is a compound represented by any one of the following Chemical Formulas 1 to 3:

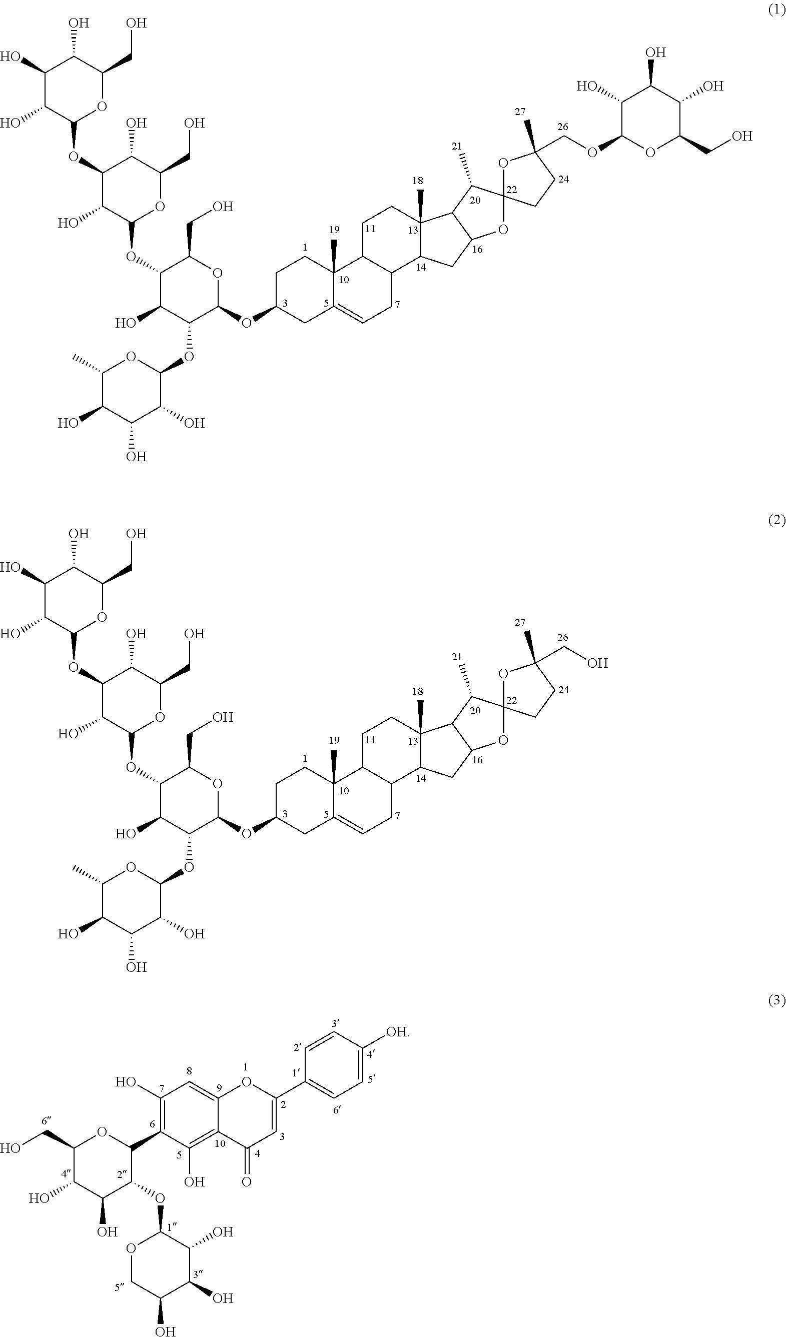

5. The method according to claim 1, wherein the composition further comprises any one or more selected from the group consisting of hyaluronic acid and collagen.

6-13. (canceled)

14. A medical device for prevention or treatment of hair loss disease comprising oat extract or a compound derived therefrom as an active ingredient.

15. The medical device according to claim 14, wherein the medical device is in the form of a microneedle patch, cream, or lotion.
